(19) United States
(12) Patent Application Publication
(10) Pub. No.: US 2017/0151152 A1
(43) Pub. Date: Jun. 1, 2017

(54) COMBINATION OF N-ACETYL GLUCOSAMINE WITH ABSCISIC ACID AND USES THEREOF

(71) Applicant: Kavita Beri, Edison, NJ (US)

(72) Inventor: Kavita Beri, Edison, NJ (US)

(21) Appl. No.: 15/367,169

(22) Filed: Dec. 1, 2016

Related U.S. Application Data
(60) Provisional application No. 62/261,839, filed on Dec. 1, 2015.

(51) Int. Cl. A61K 8/72; A61K 31/19; A61K 8/36; A61K 31/7008; A61Q 19/08; A61Q 7/00

(52) U.S. Cl. CPC A61K 8/72; A61Q 19/08; A61Q 7/00; A61K 8/36; A61K 31/7008; A61K 31/19

(57) ABSTRACT

Compositions comprising N-acetyl glucosamine and abscisic acid, a pharmaceutically acceptable salt, ester, or isomer thereof, are disclosed. The present invention also relates to the use of such a composition for skin modulation, immune modulation, and tissue growth, such as anti-aging, wound healing, skin graft healing, and stem cell maturation and signaling.

### FIELD OF THE INVENTION

Compositions comprising N-acetyl glucosamine (NAG) and Abscisic Acid (ABA), or a pharmaceutically acceptable salt, ester, or isomer thereof, are disclosed. The present invention also relates to the use of such a composition for skin modulation, immune modulation, and tissue growth, such as anti-aging, wound healing, skin graft healing, and stem cell maturation and signaling.

### BACKGROUND

Human skin is made up mainly of two main layers, namely the dermis and the epidermis that superficially covers the dermis. The dermis provides the epidermis with a solid support. It is also its nourishing element. It is made up mainly of fibroblasts and an extracellular matrix (ECM) composed mainly of collagen, elastin and a substance known as ground substance. These components are synthesized by the fibroblasts.

NAG is used in topical preparations. NAG has been shown to be effective on increasing fibroblastic activity in in-vitro studies and causing increases in gene regulation of sugar metabolism in fibroblast. Polubinska et al., Int J Cosmet Science, 2013, 35:472-476. NAG is also one of the important building blocks of hyaluronic acid, which is, a part of the ECM. NAG actions include increasing levels of hyaluronan in ECM and regulation of melanocytic genes. NAG has been shown to reduce inflammation by upregulating antioxidant enzymes and enzymes that regulate sugar metabolism (Bisset et al., J Cosmet Dermatol, 2007, 6:232-238). NAG also has a tyrosinase inhibitor effect helping with pigment reduction. The effects of topical NAG are discussed by Schlesinger et al.., Neostrata Inc. Abstract AAD, March 2015, particularly in improvement of neck rhytides.

Abscisic Acid (ABA), chemically known as (2Z, 4E)-5-[(1S)-1-hydroxy-2,6,6-trimethyl-4-oxocyclohex-2-en-1-yl]-3-methylpenta-2,4- dienoic acid is an isoprenoid stress response hormone in plants and is produced in plant stem cells that are stressed or grown in-vitro. Recent evidence shows the presence of ABA in human tissue as well. ABA may be regarded as a “universal” hormone, found across species plant/algae/human tissue with actions on a similar cellular level receptor. Sakata et al. Progress in Botany, 2013, 75:57-96. ABA has shown effectiveness in ameliorating inflammation in obesity, diabetes and cardiovascular disease models. Guri et al., Clinical Nutrition, 2010, 29:824-831. Since plant stem cell extract is derived from plant stem cells grown in-vitro, it likely contains a high concentration of ABA. Combining topical application of the NAG with plant stem cell extract enhanced anti-aging effect of laser treatment in patients. Beni et al.., J Drugs Deramatol. 2015, 14 (11):611-615. The use of combination of NAG and ABA for biological effect was hitherto unknown.

### SUMMARY

The combination of NAG and ABA, a pharmaceutically acceptable salt, ester, or isomer thereof, can be used to regulate and modify inflammatory pathways in tissue and ECM to help initiate starless tissue regeneration. The combination of NAG and ABA can be used to achieve the following:
1. Mediation of inflammatory cells or immune cells
2. Differentiation and stimulation of epidermal and mesenchymal stem cells (MSC) in the skin and in-vitro.
3. Stimulation of mesenchymal stem cells in a stimulated environment, such as with a laser
4. Synthesis of Collagen or Hyaluronic acid for ECM.
5. Implantation and homogenization of autologous stem cell derived precursor melanocyte cells vitiligo patients
6. Wound healing by mediating and controlling the action of various immune pathways, and thus facilitating “scar less” wound healing.
7. In-vitro application in mediating effective differentiation and maturation of stem cells into lineage specific cells thus aiding in-vitro organogenesis.
8. Enhance graft acceptance and quality of graft when used as a coating on biomaterial/polymeric skin grafts and bone/tissue grafts.
9. Cancer stem cell reversal

Therefore, the present invention provides compositions comprising NAG and ABA, a pharmaceutically acceptable salt, ester, or isomer thereof. The invention al so relates to the use of such a composition for skin modulation, immune modulation, and tissue growth, such as for anti-aging, anti-cancer, wound healing (both chronic and acute wounds), skin graft healing, and stem cell maturation and signaling.

### DETAILED DESCRIPTION OF THE DRAWINGS

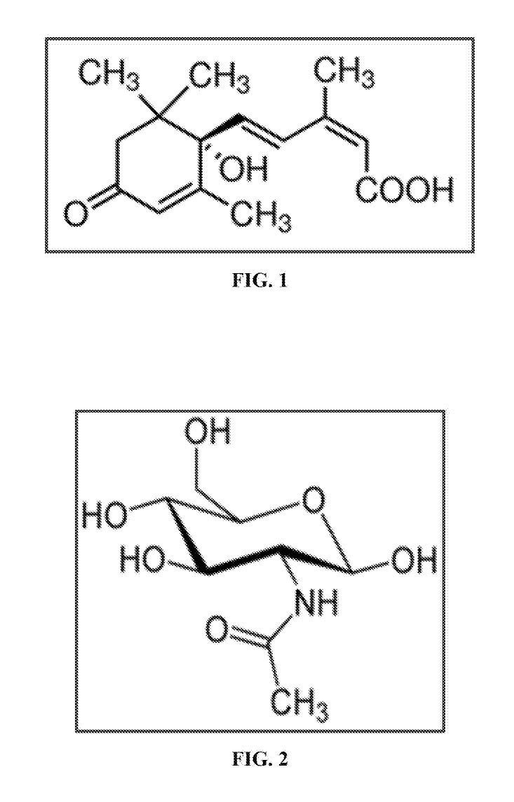

FIG. 1 shows the structural formula for Abscisic Acid (ABA)

FIG. 2 shows the structural formula for N-acetyl glucosamine (NAG).

### DETAILED DESCRIPTION

Both NAG and ABA are commercially available. The combination of NAG and ABA may be administered orally, topically or via cutaneous injection. ABA combined with NAG can be used as an ingredient in preparations, such as a gel, cream, aerosol, lotion, milk, oil, ointment, wax, mousse, paste, serum, shampoo, stick, gel capsule, tablet, or sterile injection; powder dispersion in a culture medium; as a coating on biomaterials, polymers, or scaffolding for skin grafting, wound healing, or bone grafting; or in cancer stem, cell targeted treatments which could take on several known modalities of delivery.

The amount of NAG and ABA, a pharmaceutically acceptable salt, ester, or isomer thereof to be used according to the invention depends on the desired cosmetic or therapeutic effect, and can thus vary within a wide range. A person skilled in the art can, on the basis of his general knowledge, readily determine the appropriate amounts.

Thus, and according to one preferred embodiment, the composition according to the invention comprises 0.001% and 10% by weight of NAG relative to the total weight of the composition, in particular between 0.1% and 3% by weight and more particularly between 0.5% and 5% by weight relative to the total weight of the composition. According to one embodiment, the composition according to the invention comprises ABA in an amount of between 0.4% and 10% by weight, relative to the total weight of the composition, in particular between 0.4% and 6% by weight relative to the total weight of the composition. Use of 1%, 3%, 5% of 30 μL of NAG has been reported to produce dose dependent decrease in melanin in cell cultures.

Effective concentrations of ABA may be in the range of 1 nM to 2 mM and effective concentrations of NAG may be in the range of 1 nM to 20 Mm in some preferred embodiments.

The composition according to the invention may be applied to the part of the skin, hair, nails or eyebrows, in particular to the face, the body, the neck, the hands, the hair or the scalp, or to the wound, preferably daily or several times a day. The application may, for example, be repeated every day over a variable period according to the desired effects, generally from 3 to 6 weeks, but may be prolonged or pursued continuously.

Another embodiment of the present invention is a treatment method, in particular a cosmetic or therapeutic method, for reducing or preventing the signs of ageing of the skin or hair, eyelashes, or nails, by administration to an individual, preferably a human being, of an effective amount of NAG and ABA, a pharmaceutically acceptable salt, ester, or isomer thereof. The composition or combination according to the invention is more particularly intended to be applied to the areas of the body or face of individuals showing signs of ageing of the skin, for instance wrinkles. The present invention also relates to the use, especially the cosmetic or dermatological use, of the composition or combination according to the invention for reducing and/or preventing the signs of ageing of the skin.

The composition or combination according to the invention also makes it possible in particular to stimulate the regeneration of epidermal and dermal cells, in the skin, in particular fibroblasts, especially by increasing their proliferation. This therefore provides a method, especially a cosmetic method, which is especially effective for combating the signs of chronological ageing and/or photo ageing.

According to one preferred embodiment, the use according to the present invention is intended for improving the radiance of the complexion, for reducing and/or preventing the characteristics of wrinkles and/or fine lines, for improving and/or reducing the microrelief of the skin, and/or for improving the mechanical properties of the skin and/or for increasing the resistance of the skin to mechanical attack, such as rubbing, tensions or frictions and/or for promoting skin repair.

According to another aspect of the invention, the use of the composition or of the combination according to the invention makes it possible to improve the density of the skin, its firmness and/or the cohesion of its various compartments, in particular the cohesion of the dermis with the epidermis.

The present invention also relates to the use of the composition or combination according to the invention for preventively or curatively treating is wrinkles and/or fine lines, withered skin, lack of skin elasticity and/or tonicity, thinning of the dermis, degradation of collagen fibres, flaccid skin, thinned skin and/ or any internal degradation of the skin caused by exposure to ultraviolet radiation.

The present invention also relates to the use of the composition or combination according to the invention for stimulating skin regeneration, in particular of the epidermis and/or the dermis, by means of better skin cell renewal, in particular of the epidermis and/or the dermis.

By acting on the dermoepidermal junction, keeping it folded, thus, making it possible to increase the area of the contact zone between the dermis and the epidermis, to promote exchanges between these two tissues, to reinforce their cohesion and to improve the appearance of the epidermis, the compositions according to the invention make it possible to attenuate wrinkles and/or to make the skin firm.

The composition or combination according to the present invention has the effect of improving the lipid profile of the epidermis by modifying lipogenesis and reinforcing the integrity of the skin lipids, and thus of improving the barrier function of the skin and/or the suppleness of the skin.

One particularly preferred object of the present invention is the use of the composition or combination according to the invention for improving the lipid profile of the epidermis by modifying lipogenesis, and in particular causing an increase in the synthesis of ceramides.

Another preferred object of the present, invention is the use of the composition or combination according to the invention for increasing keratinocyte and/or fibroblast proliferation, or for stimulating collagen synthesis, in particular the synthesis of procollagen I, collagen VII and collagen VI and/or XII.

Another particularly preferred object of the present invention is the use of the composition Of combination according to the invention for increasing the synthesis of tenascin and/or collagen VII.

In general, the medium in which NAG and ABA, a pharmaceutically acceptable salt, ester, or isomer thereof are included is a physiologically acceptable medium, in particular a cosmetically or pharmaceutically acceptable medium, and may be anhydrous or aqueous. It may thus comprise at least one aqueous phase and/or at least one fatty phase. The physiologically acceptable medium in which the compounds according to the invention may be employed, and also the constituents thereof, their amount, the galenical form of the composition, its mode of preparation and its mode of administration, may be chosen by a person skilled in the art on the basis of his general knowledge, as a function of the desired type of composition.

When the composition is a composition intended for topical administration, it may advantageously be in the form of aqueous or aqueous-alcoholic solutions, oil-in-water O/W) or water-in-oil (W/O) emulsions or multiple emulsions (triple: W/O/W or O/W/O), nanoemulsions, in particular O/W nanoemulsions, in which the size of the drops is less than 100 nm, aqueous gels, or dispersions of a fatty phase in an aqueous phase with the aid of spherules, these spherules possibly being polymer nanoparticles such as nanospheres and nanocapsules or lipid vesicles of ionic and/or nonionic type (liposomes, niosomes or oleosomes (as described in patent applications FR 2 709 666 and FR 2 725 369)).

The compositions are prepared according to the usual methods.

When the composition is in aqueous form, especially in the form of an aqueous dispersion, emulsion or solution, it may comprise an aqueous phase, which may comprise water, a floral water and/or a mineral water.

When the composition is an emulsion, the proportion of the fatty phase may range from about 5% to 80% by weight and preferably from about 2% to 50% by weight relative to the total weight of the composition. The oils, waxes, emulsifiers and co-emulsifiers used in the composition in emulsion form are chosen from those conventionally used in cosmetics. The emulsifier and the co-emulsifier are present in the composition in a proportion ranging from 0.3% to 30% by weight and preferably from 0.5% to 20% by weight relative to the total weight of the composition. The emulsion may also contain lipid vesicles.

When the composition is an oily solution or gel, the fatty phase may represent more than 90% of the total weight of the composition.

The oily phase may also comprise any common liposoluble or lipodispersible additive, as indicated herein below. It may especially comprise fatty substances such as waxes, pasty compounds, fatty alcohols or fatty acids. The oily phase contains at least one oil, more particularly at least one cosmetic oil. The term “oil” means a fatty substance that is liquid at room temperature.

As oils that may be used in the composition of the invention, examples that may be mentioned include: hydrocarbon-based oils of animal origin, such as perhydrosqualene; hydrocarbon-based oils of plant origin, such as liquid triglycerides of fatty acids containing from 4 to 10 carbon atoms, for instance heptanoic or octanoic acid triglycerides, or alternatively, for example, sunflower oil, corn oil, soybean oil, marrow oil, grapeseed oil, sesameseed oil, hazelnut oil, apricot oil, macadamia oil, arara oil, coriander oil, castor oil, avocado oil, caprylic/capric acid triglycerides, for instance those sold by the company Stearineries Dubois or those sold under the names Miglyol 810, 812 and 818 by the company Dynamit Nobel, jojoba oil, shea butter oil, and caprylyl glycol; synthetic esters and ethers especially of fatty acids, for instance the oils of formulae R.sup.1COOR.sup.2 and R.sup.1OR.sup.2 in which R.sup.1 represents a fatty acid or a fatty alcohol residue containing from 8 to 29 carbon atoms and R.sup.2 represents a branched or unbranched hydrocarbon-based chain containing from 3 to 30 carbon atoms, for instance Purcellin oil, 2-octyldodecyl stearate, 2-octyldodecyl erucate, isostearyl isostearate; hydroxylated esters, for to instance isostearyl lactate, octyl hydroxystearate, octyldodecyl hydroxystearate, diisostearyl malate or triisocetyl citrate; fatty alcohol heptanoates, octanoates or decanoates; polyol esters., for instance propylene glycol dioctanoate, neopentyl glycol diheptanoate and diethylene glycol diisononanoate; and pentaerythritol esters, for instance pentaerythrityl tetraisostearate, or isopropyl lauroyl sarcosinate, sold especially under the trade name Eldew SL 205 by the company Ajinomoto; linear or branched hydrocarbons, of mineral or synthetic origin, such as volatile or non-volatile liquid paraffins, and derivatives thereof, petroleum jelly, polydecenes, isohexadecane, isododecane, hydrogenated polyisobutene such, as Parleam oil, or the mixture of n-undecane (C11) and of n-tridecane (C13) sold under the reference Cetiol UT by the company Cognis; fluoro oils that are partially hydrocarbon-based and/or silicone-based, for instance those described in document JP-A-2 295 912; silicone oils, for instance volatile or non-volatile polymethylsiloxanes (PDMS) with a linear or cyclic silicone chain, which are liquid or pasty at room temperature, in particular volatile silicone oils, especially cyclopolydimethylsiloxanes (cyclomethicones) such as cyclohexadimethylsiloxane and cyclopentadimethylsiloxane; polydimethylsiloxanes comprising alkyl, alkoxy or phenyl groups, which are pendent or at the end of a silicone chain, these groups containing from 2 to 24 carbon atoms; phenyl silicones, for instance phenyl trimethicones, phenyl dimethicones, phenyltrrimethylsiloxydiphenyl-siloxanes, diphenyl dimethicones, diphenylmethyldiphenyltrisiloxanes or 2-phenylethyl trimethylsiloxy silicates, and polymethylphenylsiloxanes; or mixtures thereof.

In the list of oils mentioned above, the term “hydrocarbon-based oil” means any oil mainly comprising carbon and hydrogen atoms, and possibly ester, ether, fluoro, carboxylic acid and/or alcohol groups.

The other fatty substances that may be present in the oily phase are, for example, fatty acids containing from 8 to 30 carbon atoms, for instance stearic acid, lauric acid, palmitic acid and oleic acid; waxes, for instance lanolin wax, beeswax, carnauba wax or candelilla wax, paraffin wax, lignite wax or microcrystalline waxes, ceresin or ozokerite, and synthetic waxes, for instance polyethylene waxes and Fischer-Tropsch waxes; silicone resins such as trifluoromethyl-C1-4-alkyl dimethicone and trifluoropropyl dimethicone; and silicone elastomers, for instance the products sold under the name KSG by the company Shin-Etsu, under the name Trefil, BY29 and EPSX by the company Dow Corning, or under the name Gransil by the company Grant Industries.

These fatty substances may be chosen in a varied manner by a person skilled in the art so as to prepare a composition having the desired properties, for example in terms of consistency or texture.

The emulsions generally contain at least one emulsifier chosen from amphoteric, anionic, cationic and nonionic emulsifiers, used alone or as a mixture, and optionally a co-emulsifier. The emulsifiers are chosen in an appropriate manner according to the emulsion to be obtained (W/O or O/W). The emulsifier and the co-emulsifier are generally present in the composition in a proportion ranging from 0.3% to 30% by weight and preferably from 0.5% to 20% by weight relative to the total weight of the composition.

For W/O emulsions, examples of emulsifiers that may be mentioned include dimethicone copolyols, such as the mixture of cyclomethicone and dimethicone copolyol sold under the trade name DC 5225 C by the company Dow Corning, and alkyl dimethicone copolyols such as the lauryl dimethicone copolyol sold under the name Dow Coming 5200 Formulation Aid by the company Dow Corning, and the cetyl dimethicone copolyol sold under the name Abil EM 90® by the company Goldshmidt. A crosslinked elastomeric solid organopolysiloxane comprising at least one oxyalkylene group, such as those obtained according to the procedure of Examples 3, 4and 8 of patent U.S. Pat. No. 5,412,004 and of the examples of patent U.S. Pat. No. 5,811,487, especially the product of Example 3 (synthesis example) of patent U.S. Pat. No. 5,412,004, such as the product sold under the reference KSG 21 by the company Shin-Etsu, may also be used as surfactants for W/O emulsions.

For O/W emulsions, examples of emulsifiers that may be mentioned include nonionic emulsifiers such as oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters such as sucrose stearate; and mixtures thereof, such as the mixture of glyceryl stearate and PEG-40 stearate.

These compositions may also be O/W emulsions stabilized with particles, for instance the polymer particles described in patent FR 2 760 641, zo or crosslinked or non-crosslinked amphiphilic polymers, as described in patent applications FR 2 853 543 and FR 2 819 175.

In a blown manner, the cosmetic composition may also contain adjuvants that are common in cosmetics, such as hydrophilic or lipophilic gelling agents, hydrophilic or lipophilic active agents, preserving agents, antioxidants, solvents, fragrances, fillers, odour absorbers and dyestuffs. The amounts of these various adjuvants are those conventionally used in the cosmetics field, and range, for example, from about 0.01% to 10% of the total weight of the composition. Depending on their nature, these adjuvants may be introduced into the fatty phase, into the aqueous phase and/or into lipid spherules.

As solvents that may be used in the invention, mention may be made of lower alcohols, for instance ethanol, isopropanol, dipropylene glycol, butylene glycol and propylene glycol.

As hydrophilic gelling agents that may be used in the invention, non-limiting, examples that may be mentioned include carboxyvinyl polymers (Carbomer®), acrylic copolymers such as acrylate/alkylacrylate copolymers, polyacrylamides, polysaccharides such as hydroxypropylcellulose, natural gums and clays, and lipophilic gelling agents that may be mentioned include modified clays such as bentones, metal salts of fatty acids, for instance aluminium stearates, hydrophobic silica, ethylcellulose and polyethylene.

The compositions of the invention may contain other hydrophilic or lipophilic active agents. These active agents are chosen especially from antioxidants, demo-relaxing or dermo-decontracting agents, anti-ageing agents, anti-glycation agents, agents for stimulating the synthesis of dermal or epidermal macromolecules and/or for preventing their degradation, agents for stimulating fibroblast or keratinocyte proliferation and/or keratinocyte differentiation, agents for promoting maturation of the horny envelope, NO-synthase inhibitors, and agents for stimulating the energy metabolism of cells.

According to one alternative, the composition according to the invention may be administered by injection optionally in combination with filling products. Specifically, one of the solutions adopted for combating wrinkles and/or the loss of volume of soft tissue is the use of filling products (or filler). This filling may be achieved by using non-resorbable products, such as polyacrylamide gels or polymethyl methacrylate (PMMA) particles. However, these compounds may lead to intolerance reactions of the type such as inflammation or hypersensitivity.

The use of resorbable components, such as proteins, fats, collagen or hyaluronic acid, is preferred. However, these compounds are degraded relatively quickly in the body, which reduces their efficacy. To overcome this, more or less expensive crosslinking of these components must be performed. At the present time, the hyaluronic acid used in pharmaceutical forms or medical devices is in the form of a sodium hyaluronate gel. The monosaccharide according to the invention or the compositions containing it may also be applied by mesotherapy. Mesotherapy is a technique of treatment via intraepidermal and/or intradermal and/or subcutaneous injection of active product(s), for instance micronutrients, vitamins and/or hyaluronic acid. The compositions are administered according to this technique via injection in the form of multiple small droplets into the epidermis, the dermo-epidermal junction and/or the dermis in order especially to perform subcutaneous layering. The mesotherapy technique is especially described in the publication “Traite de mesotherapie” by Jacques Le Coz, published by Masson, 2004. Mesotherapy performed on the face is also referred to as a mesolift or a mesoglow.

Thus, another subject of the present invention may be a device, in particular a medical device, comprising an effective amount of NAG and ABA. This device may be suitable for intraepidermal and/or intradermal and/or subcutaneous injection. The combination of active agents as defined above is dissolved in a sterile medium. The said device may comprise at least one other compound, for instance at least one resorbable or non-resorbable product, such as those mentioned above, which is optionally crosslinked.

The said device may be, for example, a syringe with a needle or an injection device without a needle, such as those used in the care technique known as mesotherapy. A kit comprising a device may also be envisaged, the said kit comprising a device, in particular a syringe or an injection device, and NAG and ABA. The said kit may also comprise a needle. The said device may be in ready-to-use form, i.e. prefilled, or may need to be filled before use.

According to another aspect, the invention also relates to a cosmetic assembly comprising: i) a container delimiting at least one compartment, the said container being closed by a closing member; and ii) a composition placed inside the said compartment.

The container may be in any suitable form. It may especially be in the form of a bottle, a tube, a jar, a case, a can, a sachet or a box. The closing member may be in the form of a removable stopper, a lid, a cover, a tear-off strip or a cap, especially of the type comprising a body fixed to the container and a cap articulated on the body. It may also be in the form of a member ensuring the selective closure of the container, especially a pump, a valve or a clapper.

The container may be combined with an applicator. The applicator may be in the form of a fine brush, as described, for example, in patent FR 2 722 380, The product may be contained directly in the container, or indirectly. By way of example, the product may be arranged on an impregnated support, especially in the form of a wipe or a pad, and arranged (individually or in plurality) in a box or in a sachet. Such a support incorporating the product is described, for example, in patent application WO 01/03538.

The container may be at least partially made of thermoplastic material such as polypropylene or polyethylene. Alternatively, the container is made of non-thermoplastic material, especially glass or metal (or alloy).

The container may have rigid or deformable walls, especially in the form of a tube or a tube bottle. The container may comprise means for initiating or facilitating the distribution of the composition. By way of example, the container may have deformable walls so as to allow the composition to exit in response to a positive pressure inside the container, this positive pressure being caused by elastic (or non-elastic) squeezing of the walls of the container.

Finally, the invention relates to a therapeutic, cosmetic or dermatological treatment process comprising at least one step of administration of NAG and at least one step of administration of ABA.

The administration of NAG according to the invention may be perfoimed simultaneously with, or before or after, the administration of ABA. According to one alternative, NAG is administered first and ABA is administered second. According to another alternative, ABA is administered first and NAG is administered second. NAG and ABA may be contained separately inside two compartments, formed either by two separate containers, or inside a single device. The term “single device” means a device via which the two compartments are solidly attached. Such a device may be obtained via a process of monobloc moulding of the two compartments, especially made of a thermoplastic material. It may also result from any form of assembly, especially by bonding, welding or other click-fastening.

According to a first embodiment, the two containers are independent of each other. Such containers may be in various forms, They may especially be tubes, bottles or drums.

The containers may be fitted with a manually operated pump on which is mounted a push button for actuating the pump and dispensing the composition via at least one dispensing orifice.

Alternatively, the containers are pressurized, especially by means of a propellant, in particular a propellant gas. In this case, the container(s) is (are) equipped with a valve on which is mounted a push-button equipped with a nozzle or any other diffusion means for dispensing, the product. The propellant may be in a mixture with the composition to be dispensed or separated, especially via a piston that can slide inside the container, or via the flexible walls of a bag inside which the composition is placed.

The containers may be made of various materials including plastic, glass or metal.

Effective concentration of ABA and NAG include those reported in literature. 2 mM ABA shows in vitro cellular response. 20 mM concentration of NAG is believed to be effective. A study showing genomic changes reports use of 1%, 3%, and 5% of 30 μL of NAG. NAG produces dose dependent decrease in melanin in cell cultures.

Chemical Structure: ABA comprises a phenolic group but is relatively hydrophobic whereas NAG is highly hydrophilic. NAG facilitates the action of ABA by increasing the solubility of ABA making it available to human tissue.

### EXAMPLES

### Method of Preparing Composition

NAG is obtained from Vector Laboratories, Inc., California, and ABA is obtained from Caisson Laboratories, Inc., Utah.

### Example 1

B818 (20 mM NAG and 2 mM ABA): Dissolve 442.4 mg NAG in minimum volume of water and add 52.86 mg ABA. Solution volume is brought to 100 ml with water at 37to 38° C.

### Method of Testing Activity

### 1. Anti-Oxidant Effect on Fibroblasts

Experiments are performed on mouse dermal Fibroblasts. Cells are grown in Dulbecco's Modified Eagle's Medium (DMEM) supplemented with 10% fetal bovine serum (FBS), 100 U/ml penicillin, and 100 U/ml streptomycin. In all experiments, cells are grown to 80-90% confluence in 75 cm² culture flasks and digested with trypsin 0.05% -EDTA 0.02% solution and sub-cultured into 24-well culture plates for experiments.

H₂O₂is used to induce oxidative stress. Fibroblasts are seeded in 96-well plates in DMEM medium containing 10% FBS. After overnight culture, the fibroblsts are treated with different concentration of NAG (1 mM; 10 mM), or with different concentration of NAG and ABA composition (1 mM NAG and 0.1 mM ABA; 10 mM NAG and 1 mM ABA; B818 (20 mM NAG and 2mM ABA)), After incubation for 1h the cells are treated with 1 mM or 2 mM of H₂O₂ and incubated for 24 h. 20 μl of MTS is added to each well. After incubation for 2 h the cell proliferation (cell viability) is assayed at 490 nm using a microplate reader which reads the optical densities.

Analyze the effects of the compositions of the invention on stressed skin cells (primary Fibroblasts from mouse and human), other skin cells (Keratinocytes, Neurons, Astrocytes, Melanocytes, Mesenchymal stem cells). The following combinations are tested:
Fibroblast+B818
Fibroblast+H₂O₂
Fibroblast+B818+H₂O₂
Fibroblast+NAG
Fibroblast+NAG+H₂O₂
Fibroblast+ABA
Fibroblast+ABA+H₂O₂

Run dose-response experiments to define the right amount of H₂O₂ (0.01, 0.05, 0.1, 0.5, 1, 5 and 10 mM from 30% H₂O₂, stock solution) to be used to kill 40-50% of the fibroblasts after 48 hours.

### 2. Synthesis of Hyaluronic and Collagen

Fibroblasts are seeded into culture plates and grown until they reached confluence. Afterwards, 24 h incubation is started in culture medium (control) or in medium supplemented with NAG (1, 2.5, 5 and 10 mM) or with compositions of the invention (1 mM NAG and 0.1 mM ABA; 2.5 mM NAG and 0.25 mM ABA; 5 mM NAG and 0.5 mM ABA; 10 mM NAG and 1 mM ABA; and B818 (20 mM NAG and 2 mm ABA)). All conditions are duplicated. At the end of incubation, while still in presence of control medium or medium supplemented with NAG or compositions of the invention, the synthesis of hyaluronic and collagen are studied as below:

Run quantitative RT-PCR, ELISA to detect modifications of collagen type stimulated: Test the expression and synthesis of collagen; Collagen type I and III (primers; collagen Type I, chain α1, collagen Type III, chain α1) and observe modification in the ratio Collagen type I:Collagen type III.

Concentration of hyaluronic in supernatant is measured with quantizing ELISA kit from R&D system. See https://resources.rndsystems.com/pdfs/datasheets/dhya10.pdf

The total amount of soluble collagen is assayed using the soluble collagen assay kit MET-5016 from Cell Bio labs Inc. See http://www.cellbiolabs.com/sites/default/files/MET-5016-soluble-collagen-assay-kit.pdf

### 3. MSC (Mesenchymal Stem Cell) Stimulation Source (Bone Marrow/Hair Follicle/Cord Blood)—Test Stimulation of MSC—Maturation and Proliferation

McCoy's medium (Lonza, Milan, Italy, http://www.lonza.com) (or a different source) is supplemented with 10% fetal bovine serum (FBS; Invitrogen, Milan, Italy, http://www.invitrogen.com) and with 100 U/ml penicillin plus 100 μg/ml streptomycin. After 48 hours of culture, the medium is removed, adherent cells are washed once with phosphate-buffered saline, and fresh medium is added to each flask. Cells are maintained at 37° C. in a humidified atmosphere with 5% CO₂, and half of the medium is changed every 3 days. Cell confluence is usually reached in 2 weeks, this being considered passage 0: cells are then expanded (1:4) and cultured as described. Cells from passages 2-4 are used for all experiments.

### 4. Effect on ADPRC Activity and CD38 Expression in Human MSC

The clonogenic assays (colony forming units fibroblasts [CFU-F]) is set up with freshly isolated BM-MNC (Bone Marrow-Mono Nuclear Cells). The cells are stimulated with the following compounds for the 1st week of culture: 1, 20 , 100 mm NAG , B818 (20 mm NAG and 2 mm ABA) or 10 μM, 0.5 mM, 1 mM ABA, the latter without or with 50 μM 8-bromo-cADPR (8-Br-cADPR). After 2 weeks, cells are stained with crystal violet, and the number of colonies is counted.

For the proliferation assay, cells are plated at a density of 3×10³ cells per well in 96-well plates. After 24 hours, the same compounds used in the CFU-F assay are added, in quadruplicate. After 3 days of culture, 3-(4,5-dimethylthiazol-2-yl)-2,5-diphenyltetrazolium test is performed to evaluate the extent of cell proliferation (http://onlinelibrary.wiley.com/doi/10.1634/stemcells.2008-0488/full#bib19). To explore possible changes in cell phenotype, fluorescence-activated cell sorting analyses are performed on MSC cultured for 1 week in the presence or absence of ABA, NAG and B818. The surface antigens test is performed.

### 5. Effect on Wound Healing Cytokines

Repeat above cellular protocols and use individual ELISA measuring kits for the following cytokine and growth factor assays:
Cytokines, epidermal growth factor (EGF) family, transforming growth factor beta (TGF-β) family, fibroblast growth factor (FGF) family, vascular endothelial growth factor (VEGF), granulocyte macrophage colony stimulating factor (GM-CSF). platelet-derived growth factor (PDGF), connective tissue growth factor (CTGF), interleukin (IL) family, and tumor necrosis factor-α family.

B818 Potential wound healing effect of B818 application or B818 application combined with laser can be assessed directly on keratinocyte cell culture (in vitro scratch assay). Fibroblast scaffold wound healing models may be used.

### 6. Wound Healing, Skin Regeneration, Scar Less Healing: Partial and Full Thickness Skin Biopsies:

Test pig skin using punch biopsy. Full thickness skin punch—5 mm. Vaseline is applied on control site and B818 is applied on test site within 2 hours, of wound. Repeat punch biopsy on wound sites in 1 week to compare on histology haematoxylin and eosin stain for cellular changes.

### 7. Effect on Hair Growth: Hair Follicle Regeneration

Observe through Ramen Spectrometry for penetration of the composition in hair follicle bulge after periodic application of B818 composition scalp skin. Observe cellular changes to hair follicular cells including maturation, growth and other activity.

1. A compositions comprising:
N-acetyl glucosamine, and
abscisic acid, or a pharmaceutically acceptable salt, ester, or isomer thereof.

2. Use of the composition of claim 1 for skin modulation, immune modulation, or tissue growth.

3. Use of the composition of claim 1 as an anti-aging agent,

4. Use of the composition of claim 1 for wound healing, skin graft healing, skin regeneration, scar less healing, hair growth, or stem cell maturation and signaling.

5. Use of the composition of claim 1 for hair follicle regeneration.
